(19) United States
(12) Patent Application Publication
(10) Pub. No.: US 2008/0221536 A1
(43) Pub. Date: Sep. 11, 2008

(54) Device for Carrying Out the Minimally Invasive Withdrawal of Blood from Animals by Using Blood-Sucking Assassin Bugs

(75) Inventors: Ruth Thomsen, Berlin (DE); Christian Voigt, Berlin (DE)

Correspondence Address: Pearl Cohen Zedek Latzer, LLP, 1500 Broadway, 12th Floor, New York, NY, 10036, US

(73) Assignee: FORSCHUNGSVERBUND BERLIN E.V., BERLIN GERMANY (DE)

(21) Appl. No.: 10/586,472

(22) PCT Filed: Jan. 20, 2005
(86) PCT No.: PCT/DE05/00087

§ 371 Date: Jul. 18, 2006

(30) Foreign Application Priority Data
Jan. 20, 2004 (DE) 10-2004-004-066.4

(51) Int. Cl. A61B 5/15

(52) U.S. Cl. 604/322

(57) ABSTRACT

The invention concerns a device for carrying out the minimally invasive withdrawal of blood from animals by using blood-sucking assassin bugs. Blood samples are required within the scope of many testing arrangements, hence blood withdrawals are one of the most common interventions. In all known methods, a tapping of a blood-stream is carried out using a medical instrument. These blood withdrawals are often complicated and are subject to strict regulations. Furthermore, the animals have to be anesthetized sometimes. Assassin bugs have also been used for withdrawing small quantities of blood. The blood of bats stored in the abdomen of the assassin bugs is removed for tests. However, it is often difficult to locate the bug after it has sucked blood because it quickly flees to dark places. The inventive device is comprised of at least one receptacle (1), which has at least one perforated outer wall (1.1) and in which the assassin bug W can be positioned on the animal of interest (F) whereby enabling the assassin bug (W) to suck blood therefrom. Afterwards, the assassin bug (W) is fixed at a location on the perforated outer wall (1.1) inside said receptacle (1) by means of a moving intermediate wall (1.2) so that the blood sucked out from the animal and stored in the abdomen can be removed from the assassin bug (NV) via a cannula.

The invention concerns a device for carrying out the minimally invasive withdrawal of blood from animals by using blood-sucking assassin bugs, for research, testing and diagnostic purposes.

Blood samples are required within the framework of many testing procedures; accordingly, blood withdrawals are among the more common interventions. On one hand, the withdrawal of blood in trials on animals is required for the authorization of medications, in order to confirm the harmlessness of the content of said medications for later use on human beings. On the other hand, the withdrawal of blood is also required in ordinary veterinary examinations, in order to discover the causes of an animal's illness.

While the withdrawal of blood is not problematic when large animals are concerned, because their blood vessels are easily accessible and contain large volumes of blood, the withdrawal of blood from smaller animals poses a difficulty, due to the smaller quantities of blood and the extremely fine and often poorly accessible blood vessels. As the possibilities for withdrawing blood are limited, the legal limit of strain which may be imposed on laboratory animals in the course of laboratory trials is generally exhausted. Aside from the fact that the death of the animal trial subjects must be taken into account and, in some cases, is intentionally caused (terminal withdrawal of blood from the chambers of the heart), there is an additional problem: diagnosis and kinetic studies require blood withdrawals on a continuous basis, in order to be able to determine changes in the effective ingredient or reductions in the concentration of administered medications over time.

In pets, the withdrawal of blood is difficult, because, in view of the atmosphere in the veterinary clinic, the animal may be expected to be restless and/or to show resistance, which can only be overcome by force or by anesthesia. Both of these, however, affect the animal's blood count, so that the usual methods for withdrawal of blood cannot guarantee the exclusion of outside influences on the blood count. Generally speaking, blood is withdrawn by means of special instruments, such as a syringe or a capillary tube. These are used to puncture a vein which is accessible from the outside in order to withdraw blood. As a function of the size of the animal in question, however, the volume of the blood sample to be taken becomes more limited and the access to blood vessels becomes more difficult. The size of the sample volume must be selected accordingly, taking into account the frequency of sampling, the limits of the pathophysiological compensation mechanism and the regeneration time. The quantity of blood which may be taken from an animal, however, is not only-dependent on the maximum blood volume which may be drawn (for example, in a rat with a body weight of 100 g and a total blood volume of 5 ml, that quantity is 1 ml, or 20%), but also on the blood withdrawal technique and the skill of the person performing the intervention. For this reason, blood withdrawal techniques must be selected which do not cause too much stress for the animal in question and which guarantee a constant in sufficient quantity of blood. This means that, when withdrawing blood from small animals (with a body weight of less than approximately 3 kg), only blood samples of less than 1 ml may be taken; this type of sampling, by means of the usual blood withdrawal techniques, may necessitate puncturing the heart, piercing or often penetrating the veins, or repeated removal of pieces of the animal's tail.

The possibility of withdrawing blood by piercing the veins of rats' tongues has also been described [German Federal Ministry of Veterinary Medicine: “Blood Withdrawal in Laboratory Rodents and Rabbits for Experimental Purposes”, Information on Animal Protection 3.02, page 7]. In this process, the tongue of the laboratory animal is gripped with the fingers and pierced by means of a thin needle. Admittedly, this procedure can be repeated a number of times. The animal, however, will subsequently have to be sacrificed if it refuses food for more than one day; as a result, reproducible and significant experimental results often cannot be guaranteed.

In addition, the puncturing is carried out under anesthesia; this leads to a change in the composition of the blood and represents an additional factor which precludes the reproducibility of the experiment.

Retrobulbar blood withdrawal, in which the plexus of veins behind the eye is punctured, is an additional technique for withdrawing blood. The anatomical proximity to the eye, in addition to the practical difficulty of determining the precise location of the optic vein, also presents a normative problem, because, in cases involving repeated sampling, errors in manipulation can lead to hematoma of the eye or blindness. Pursuant to §§13 I, 13a II of the German Animal Protection Law, experiments on animals involving stress are to be kept to the unavoidable minimum and/or require a permit. In this context, as set forth in the description given by the Tierärztliche Vereinigung für Tierschutz e.V. (German Association of Veterinarians for Animal Protection), “Notes on Blood Withdrawal from Small Laboratory Animals”, p. 3, this withdrawal technique must be practiced under anesthesia, and the quantities of blood to be obtained through the use of this technique are limited, inconsistent and contaminated with foreign matter.

In all methods known from prior art, a medical instrument is used to puncture a blood vessel and thereby to withdraw blood. The volumes which may be withdrawn in this way, however, are small; moreover, exact localization of the blood vessels from the outside cannot always be guaranteed. In addition, as described in the methods set forth above, it is sometimes necessary to anesthetize the animals.

A completely different method for withdrawing blood from small animals has been described by O. v. Helversen, M. Volleth and J. Núnez in “A new method for obtaining blood from a small mammal without injuring the animal: use of Triatomid bugs”, Experientia 42 (1986), pp. 809 and 810. For the withdrawal of small quantities of blood from small mammals, assassin bugs were used, which, for example, sucked blood from bats. The bats' blood, which was stored in the abdomen of the assassin bugs, was withdrawn from the bugs by means of cannulas known from prior art and could be used for various tests. Often, however, it proved difficult to hold the bugs in one place after they sucked blood, because they quickly escaped to dark places. C. C. Voigt, M. Fassbender, M. Dehnhard, G. Wibbelt, K. Jegenow, in “Validation of a minimally invasive blood-sampling technique for the analysis of hormones in domestic rabbits, Oryctolagus cuniculus (Lagomorpha)”, General and Comparative Endocrinology 135 (2004), pp. 100-107, compared the changes in the blood composition, on the basis of changes in the concentration of hormones in the blood, in the performance of blood sampling through the use of cannulas by contrast to blood sampling through the use of assassin bugs. On the basis of cortisol measurements, this series of experiments established that the stress factor of laboratory animals subjected to blood sampling through the use of cannulas was greater than that of laboratory animals subjected to blood sampling through the use of assassin bugs. In addition, blood sampling through the use of assassin bugs does not change the blood composition and constitutes a process for blood withdrawal from animals which is less stressful than the blood withdrawal technique through the use of cannulas.

The objective of the invention is to overcome the negative effects of the usual, conventional blood withdrawal techniques. On one hand, the invention should guarantee that, in small animals which, to date, are harmed by conventional blood withdrawal methods, a gentle blood withdrawal technique can be accomplished, and furthermore, can be repeatedly used without distorting the results. On the other hand, through the use of assassin bugs, it should enable blood samples to be taken from species of animals from which, to date, it has not been possible to withdraw blood. In addition, it should be easy to hold the bug in one place after its meal of blood, in order to prevent it from escaping and to enable blood to be drawn from its abdomen without problems.

This objective is solved, according to the invention, by means of a device with the characteristics of Claim 1, whereby said device consists of at least one receptacle, which exhibits at least one perforated outside wall, and within which the assassin bug can be positioned on the animal in such a way that the assassin bug is able to suck blood from it. Subsequently, the assassin bug can be held in place within the receptacle, by means of a movable partition, which holds it in place against a location on the perforated outside wall, so that the blood which has been sucked from the animal and is now stored in the abdomen of the assassin bug can be directly and rapidly removed from the bug by means of a cannula.

In a preferred embodiment, the receptacle consists of a hollow cylinder which is perforated on one of its end surfaces, in such a way that the assassin bug located within the receptacle can be positioned against the animal. After the assassin bug has sucked blood from the animal through the perforated end surface, the assassin bug, by means of a piston rod on the opposite end surface and the piston fastened thereto, which constitutes the movable partition, can be gently held in place against a location on the perforated outside wall, so that the blood can be withdrawn from the bug's stomach.

In an additional embodiment, the device consists of two adjoining receptacles, whereby the assassin bug is located in the first receptacle and the animal to be sampled in the second receptacle. A separating wall, at least part whereof is perforated, is located between these two receptacles. The animal is held in place by means of a movable disk against the perforated separating wall, in such a way as to enable the assassin bug to suck blood. The first receptacle, in which the assassin bug is located, exhibits—similarly to the first preferred embodiment—a movable partition by means of which the assassin bug can be held in place against the perforated end surface, by means of the movable piston rod.

A further embodiment of the device consists of an approximately spherical receptacle with at least one perforated outside wall and an elastic, deformable partition, whereby the partition, as a function of its material properties, can change its position and form through the effect of forces exerted from the outside, and is thus suitable for holding the bug in place between the perforated outside wall and the partition. The change in form and position of the partition can take place, for example, by means of a mechanical process. At the end of the sucking process, the bug can be held in place in the spherical receptacle for the purpose of blood withdrawal.

A deformable receptacle with a mesh-like fabric structure could conceivably serve as an additional embodiment. By means of a flexible holding device, the assassin bug can be positioned in the flexible receptacle against the animal, for the purpose of blood withdrawal, so that it can suck blood from it and, at the end of the sucking process, can be removed from the animal by means of the flexible holding device. By folding the mesh-like fabric structure together, the bug can then be held in place in the receptacle. The withdrawal of blood from the bug is implemented by means of a cannula, similarly to the embodiment described above.

The device for withdrawal of blood through the use of assassin bugs, by comparison to prior art, has the advantage that the assassin bug, which is capable of autonomous blood withdrawal, is kept in a closed receptacle, so that, by means of a perforated partition, it can reach certain body areas of the animal under test, in order to withdraw blood from it, and can subsequently be held in place for the purpose of blood sampling. If the bug finds a vein, it bites the animal and begins the sucking process. The assassin bug identifies the animal by its body temperature as soon as it gets close enough to it (approximately 50 cm) and bites as soon as it is able to do so. Generally speaking, the complete sucking process takes between four and eight minutes and can be repeated according to the quantity of blood required. The repetition can be performed rather frequently, through the use of the bugs. The bite and the sucking are not noticed by the animal, so that it need not be assumed that the animal is in any way compromised to the application of the bug. No wound remains on the animal.

In addition, the blood of the laboratory animal is basically not changed by the storage in the abdomen of the bug, as described in Experientia 42 (1986), “A new method for obtaining blood from a small mammal without injuring the animal: use of Triatomid bugs”, by O. v. Helversen, M. Volleth and J. Núnez. Furthermore, multiple blood withdrawal, with differentiated defined volumes, is possible, through the use of different developmental stages (i.e. sizes) of assassin bugs. The quantity of blood withdrawn can be up to 4 ml, according to the size and developmental stage of the bug. The blood is available for examination immediately after extraction from the blood. The composition and molecular properties of the blood are basically not changed by the withdrawal and storage in the abdomen of the bug. These circumstances are described in “Validation of Non-Invasive Blood Sampling Technique for Double-Labeled Water Experiments” by C. Voigt, O. v. Helversen, R. Michener and T. Kunz in Journal of Experimental Zoology 296A: 87-97 [88] (2003). That article proves that the sampled blood is almost completely free of contamination by substances belonging to the bug. This means that examination of the blood thereby obtained, with no residues or foreign matter, may be performed for the benefit of many areas of veterinary medicine, science and pharmacological research.

The invention is explained in greater detail below by means of various embodiments.

The appropriate drawings represent:

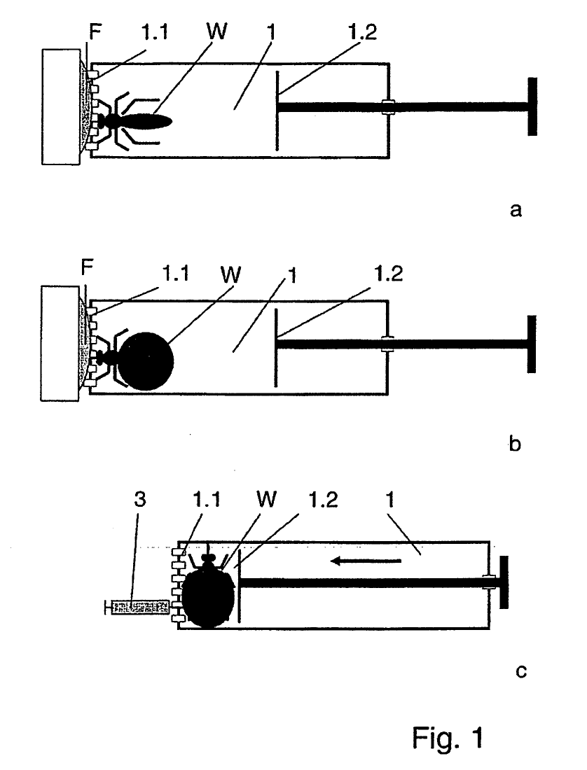

FIG. 1: Embodiment of the device as a hollow cylinder.

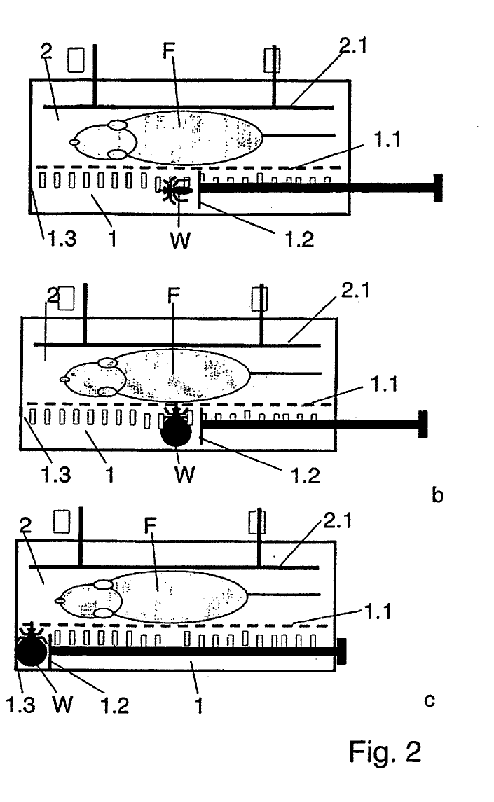

FIG. 2: Embodiment of the device with two adjacent receptacles.

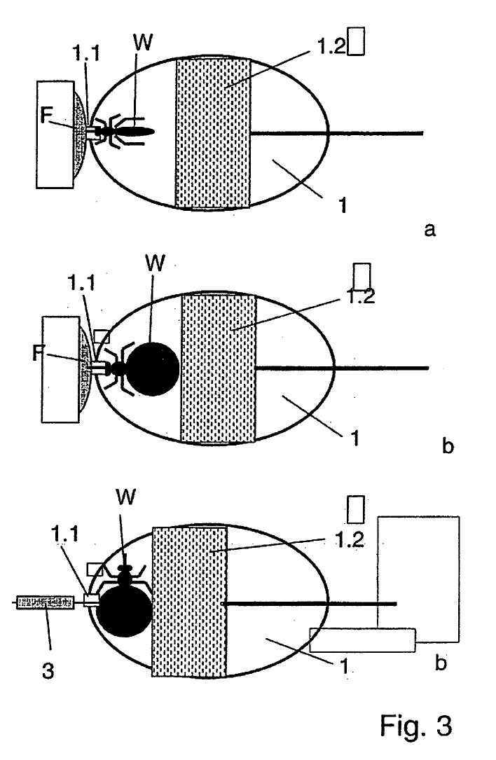

FIG. 3: Embodiment of the device with a spherical receptacle.

In the embodiment shown in FIG. 1, the receptacle 1 of the device according to the invention consists of a hollow cylinder, with one perforated end surface 1.1. On the opposite end surface is a piston rod with a piston attached thereto, whereby said piston constitutes a movable partition 1.2.

As shown in FIG. 1a, the assassin bug W is located in the aforesaid receptacle 1, between the perforated end surface 1.1 in the movable partition 1.2. The receptacle 1 is now positioned with its perforated end surface 1.1 against the animal F in such a way that the bug can reach the animal F for the purpose of blood withdrawal, and, by means of its sucking proboscis, can go through the perforated end surface 1.1 and suck the blood from the animal F.

FIG. 1b shows the bug W in the receptacle 1, against the perforated outside wall 1.1 shortly before the end of the sucking process, as may be seen by the swollen abdomen of the bug W.

It may be seen in FIG. 1c that the bug, following the end of the sucking process, is held in place by means of a movable partition 1.2, which is pressed against the perforated end surface 1.1 by means of a piston rod on the opposite end surface, whereby the freedom of movement of the bug is limited to nearly zero. By means of a syringe and a cannula 3, the blood can then be withdrawn from the swollen abdomen of the bug W which is being held in place.

An additional embodiment of the device according to the invention is shown in FIG. 2. In this embodiment, the device according to the invention consists of two adjacent receptacles 1, 2, whereby the at least partially perforated outside wall of receptacle 1 constitutes the common separating wall 1.1 between it and the second receptacle 2. The assassin bug W is located in the first receptacle 1 and the animal F in the second receptacle 2.

FIG. 2a shows that the animal F—in this case, a small mammal from which the blood is to be withdrawn—is held in place -against the perforated separating wall 1.1 by means of a movable disk 2.1, in such a way that the assassin bug W can bite it and suck blood.

FIG. 2b again shows the bug W in the first receptacle 1 against the perforated separating wall 1.1 shortly before the end of the sucking process, as may be seen by the swollen abdomen of the bug W.

FIG. 2c shows, following the conclusion of the blood sampling by the assassin bug W, how the bug, similarly to the first preferred embodiment according to FIG. 1, is held in place against a second perforated outside wall 1.3 by means of the movable partition 1.2. Again, by means of a syringe and a cannula 3, the blood sucked from the animal F by the assassin bug W, which is now held in place, can be removed from the bug W.

As shown in FIG. 3, a further embodiment of the device consists of an approximately spherical receptacle 1 provided with a partially perforated outside wall 1.1 and a movable, elastic, deformable partition 1.2. The partition 1.2 is, for example, a foam rubber body, whose entire circumference lies approximately in the middle of receptacle 1, against the outside wall of the spherical receptacle. The assassin bug W is located in one part of the spherical receptacle 1, between the partition 1.2 in the perforated outside wall 1.1. Following the end of the sucking process, the bug W can be held in place, for the purpose of removal of the blood by means of a syringe and a cannula 3, in the spherical receptacle 1, between the perforated outside wall 1.1 in the movable partition 1.2. This can take place, for example, by implementing an initial mechanical displacement of the deformable partition 1.2 so that the flexible partition 1.2 lies against the bug W, and then implementing a further displacement of the deformable partition 1.2 so that the bug W is held in place against the perforated outside wall 1.1. This spherical or ovoid receptacle is preferably suitable as a device for withdrawal of blood from birds, whereby the receptacle is placed in the bird's nest.

In addition, a flexibly deformable receptacle 1 with a mesh-like fabric structure can serve as a variant embodiment of the device according to the invention, which is positioned against the animal F by means of a flexible holding device, which consists, for example, of a fishing rod and the fishing line fastened to it, in such a way that the bug can suck the blood from the animal F. Following the end of the sucking process, the flexibly deformable receptacle 1, with the assassin bug W contained therein, is removed from the animal F by means of the fishing rod and fishing line. The bug W can be held in place, as required for withdrawal of blood from it—for example, by folding together the mesh-like fabric structure of receptacle 1. The removal of the blood from the assassin bug W is accomplished by means of a cannula 3 and a syringe, similarly to the process described in the variant embodiments set forth above.

1.-5. (canceled)

6. A device for carrying out the minimally invasive withdrawal of blood from an animal by using blood-sucking assassin bugs, the device comprising:
a receptacle comprising a perforated outside wall, and within which an assassin bug is positioned such that the assassin bug is able to suck blood from the animal, the receptacle comprising a movable partition to hold the assassin bug in place against a location on the perforated outside wall, such that blood can be removed from the abdomen of the assassin bug using an injection syringe.

7. Device according to claim 6, wherein the receptacle is a hollow cylinder and the perforated outside wall is formed by one end surface of the receptacle, and wherein the receptacle comprises a piston rod on the opposite end surface on which the movable partition is arranged.

8. Device according to claim 6, comprising a second receptacle connected to the receptacle, wherein the at least partially perforated outside wall of the receptacle forms a common separating wall between the receptacle and the second receptacle, wherein the assassin bug is located in the receptacle and the animal is held against the perforated common separating wall in the second receptacle by a movable disk, so that the assassin bug can be positioned against the animal and can withdraw blood from the animal, and, once the blood has been withdrawn, the assassin bug can be held in place against a second perforated outside wall by the movable partition.

9. Device according to claim 6, wherein the receptacle is approximately spherical and wherein the partition is elastic and deformable.

10. Device according to claim 6, wherein the receptacle is deformable and comprises a mesh-like fabric structure, by which the assassin bug can be positioned against the animal through the use of a flexible holding device.
